(19) United States
(12) Patent Application Publication
(10) Pub. No.: US 2024/0008947 A1
(43) Pub. Date: Jan. 11, 2024

(54) INTUBATION SHIELD

(71) Applicant: The Children's Medical Center Corporation, Boston, MA (US)

(72) Inventors: Farokh R. DEMEHRI, Boston, MA (US); Heung KIM, Boston, MA (US); Robert CRUM, Boston, MA (US); Brianna SLATNICK, Boston, MA (US); Kyle WU, Boston, MA (US); Alexander YANG, Boston, MA (US); Nathaniel TIGHE, Boston, MA (US); Pete KOVATSIS, Boston, MA (US); Jonathan DURGIN, Boston, MA (US)

(21) Appl. No.: 18/042,292

(22) PCT Filed: Aug. 20, 2021
(86) PCT No.: PCT/US21/71243

§ 371 Date: Feb. 20, 2023

Related U.S. Application Data
(60) Provisional application No. 63/068,686, filed on Aug. 21, 2020.

(51) Int. Cl. A61B 90/00; A61B 46/20

(52) U.S. Cl. CPC A61B 90/05; A61B 46/20; A61M 16/0488

(57) ABSTRACT

A device for shielding a user from a patient during an intubation procedure or other procedure comprises a base, a shield, and a flexible drape. The shield is coupled to the base, and extends vertically and horizontally relative to the base. The flexible drape is coupled to or overlaid on the shield, such that an interior space is defined by the shield and the flexible drape. The flexible drape includes at least one opening to allow access into the interior space.

### CROSS-REFERENCE TO RELATED APPLICATIONS

This application claims the benefit of, and priority to, U.S. Provisional Patent Application No. 63/068,686 filed on Aug. 21, 2020, which is hereby incorporated by reference herein in its entirety.

### TECHNICAL FIELD

The present disclosure is related to shields for use during an intubation procedure.

### BACKGROUND

When a patient is intubated, a healthcare worker often stands in close proximity to the patient, including the patient's mouth and nose. This close proximity of the healthcare worker increases the risk that the healthcare worker will be exposed to bodily fluids, such as blood, respiratory droplets, etc. These bodily fluids can carry bacteria, viruses, etc. and thus healthcare workers are at risk of being exposed to such bacteria, viruses, etc. when intubating a patient. Thus, new devices and methods are needed for protecting healthcare workers during intubation procedures and other procedures.

### SUMMARY

According to aspects of the present disclosure, a device for shielding a user from a patient during an intubation procedure or other procedure comprises a base, a shield, and a flexible drape. The shield is coupled to the base, and extends vertically and horizontally relative to the base. The flexible drape is coupled to or overlaid on the shield, such that an interior space is defined by the shield and the flexible drape. The flexible drape includes at least one opening to allow access into the interior space.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings.

FIG. 1A is a top view of a first implementation of a device for use during intubation procedures to protect healthcare workers from respiratory droplets, according to aspects of the present disclosure.

FIG. 1B is a side view of the device of FIG. 1A, according to aspects of the present disclosure.

FIG. 2 is a perspective view of the device placed over a patient, according to aspects of the present disclosure.

FIG. 3A is a perspective view of a first implementation for allowing a user to access an interior space of the device of FIG. 1A, according to aspects of the present disclosure.

FIG. 3B is a perspective view of a second implementation for allowing a user to access an interior space of the device of FIG. 1A, according to aspects of the present disclosure.

FIG. 4A is a perspective view of a height-adjustable post for use with the device of FIG. 1A, according to aspects of the present disclosure.

FIG. 4B is a view of a first mechanism for locking the height-adjustable post of FIG. 4A, according to aspects of the present disclosure.

FIG. 4C is a view of a second mechanism for locking the height-adjustable post of FIG. 4A, according to aspects of the present disclosure.

FIG. 4D is a view of a hydraulic mechanism to implement the height-adjustable post of FIG. 4A, according to aspects of the present disclosure.

FIG. 5A is a top view of a second implementation of a device for use during intubation procedures to protect healthcare workers from respiratory droplets, according to aspects of the present disclosure.

FIG. 5B is a perspective view of the shield of FIG. 5A decoupled from the post of FIG. according to aspects of the present disclosure.

FIG. 6 is a perspective view of the device of FIG. 5A with a flexible drape laid thereon, according to aspects of the present disclosure.

FIG. 7 is a data plot illustrated the removing of unwanted particles from the device of FIG. 5A, according to aspects of the present disclosure.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements, and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

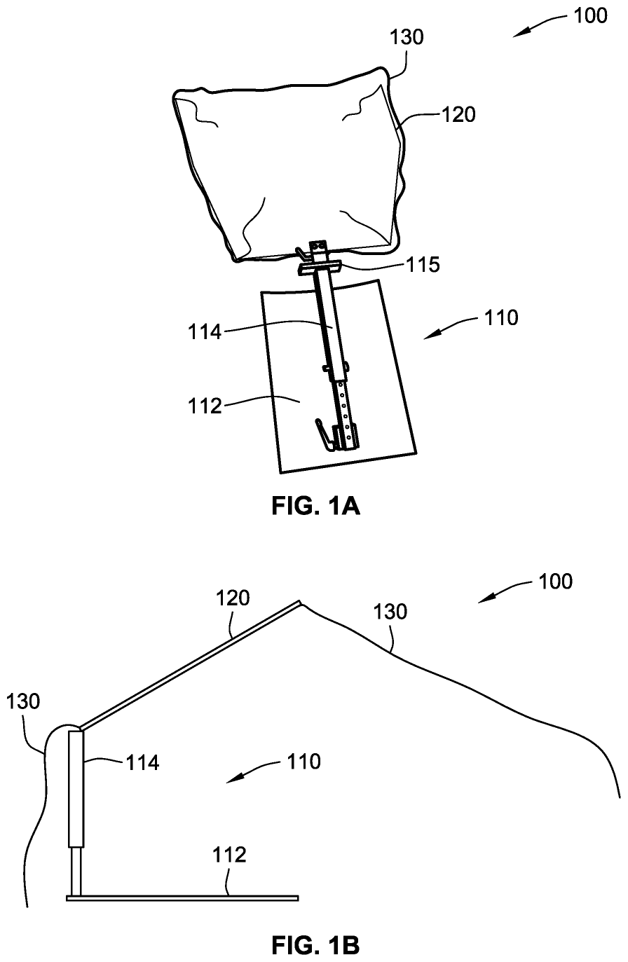

FIG. 1A and FIG. 1B show a device 100 that can be used during intubation procedures to protect healthcare workers from respiratory droplets (or other bodily fluids) that may be emitted by a patient during the intubation procedure. The device 100 may be used during other procedures as well. FIG. 1A shows a view of the device 100 in a collapsed position (which allows the device 100 to be easily stored), while FIG. 1B shows a side view of the device 100 in a deployed position. The device 100 includes a base 110, a shield 120, and a flexible drape 130. The shield 120 is coupled to the base 110, while the drape 130 is coupled to or overlaid on the shield 120. The shield 120 is generally transparent.

The base 110 generally includes a baseplate 112 and a post 114. The baseplate 112 is coupled to one end of the post 114, while the shield 120 is coupled to the opposite end of the post 114. As shown in FIG. 1A, when the device 100 is in a collapsed position, the baseplate 112, the post 114, and the shield 120 are all generally positioned in the same plane. However, the baseplate 112 and the shield 120 are both movably coupled to the post 114. Thus, as shown in FIG. 1B, when the device 100 is in the expanded position, the baseplate 112 is positioned generally horizontally, the post 114 is positioned generally vertically, and the shield 120 is positioned at an angle such that the shield 120 extends both vertically and horizontally from the post 114. In some implementations, the shield 120 is positioned at an angle of about 45° relative to the baseplate 112 when the device 100 is in the expanded position (e.g., relative to horizontal). In other implementations, the shield 120 is positioned at an angle of between about and about 60° relative to the baseplate 112 when the device 100 is in the expanded position. As shown in FIG. 1B, the drape 130 can extend over the top of the shield 120 and away from the base 110. The drape 130 can also extend on the opposite side of the post 114 of the base 110. The drape 130 will also extend on either side of the device 100 as well.

In some implementations, the upper end of the post 114 includes an attachment point 115 that the shield 120 can be attached to. The attachment point 115 is generally a structure that defines a slot configured to receive a portion of the shield 120. The post 114 may also include a locking structure (such as a clip, a clamp, or some other structure) that is configured to releasably lock the shield 120 to the post 114. The shield 120 can thus be secured to the post 114 during use, but can also be unlocked and removed from the post 114 if the shield 120 requires maintenance is to be discarded.

In some implementations, the angle of the shield 120 relative to the post 114 when in the deployed position can be adjusted. In one example, the angle of the shield 120 can be adjusted relative to the attachment point 115, which is fixed to the post 114. In another example, the angle of the attachment point 115 can be adjusted relative to the post 114, while the shield 120 is fixed to the attachment point 115. The attachment point 115 can be configured to lock the angle of the shield 120 relative to the attachment point 115, or lock the angle of the attachment point 115 relative to the post 114. In other implementations, a pair of angular support beams can be adjustably coupled to the shield 120 and either the post 114 or the attachment point 115, to adjust and lock the angle of the shield 120.

Further, the device 100 can generally be locked in both the collapsed position (FIG. 1A) and the expanded position (FIG. 1B). The baseplate 112 can thus be rotated relative to the post 114, and locked into a position that is generally parallel to the post 114 (FIG. 1A), or a position that is generally perpendicular to the post 114 (FIG. 1B). The shield 120 can also be locked into a position that is generally parallel to the post 114 (FIG. 1A), or a position that is generally perpendicular to the post 114 (FIG. 1B).

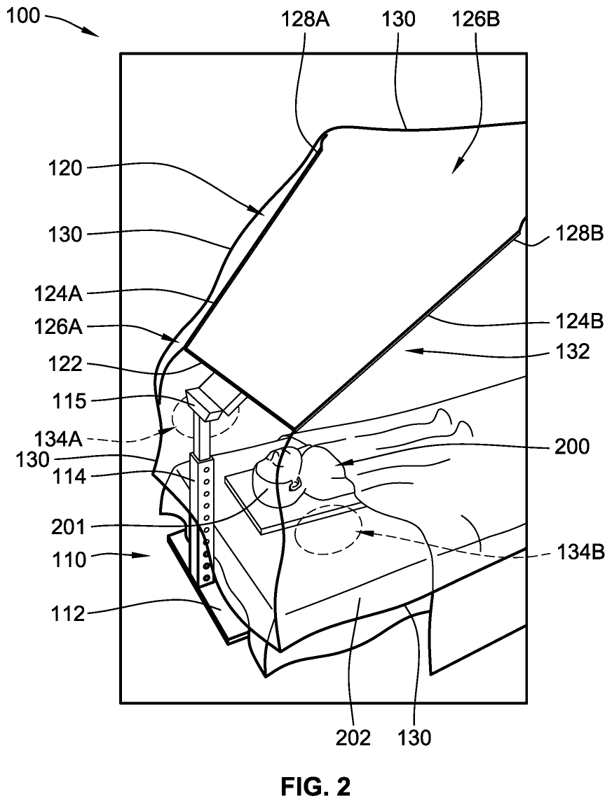

FIG. 2 shows the device 100 in use with a patient 200 lying on a bed 202. As shown in FIG. 2, when the device 100 is in the expanded position, the baseplate 112 can extend underneath the bed 202, to secure the device 100 in place. The baseplate 112 could also extend beneath the head of the patient 200, beneath a portion of the frame of the bed 202, or be attached to or interact with some other structure. The post 114 rises above the patient 200, and the shield 120 extends out over the patient 200.

In the illustrated implementation, the shield 120 is formed from crossmember 122 and two legs 124A, 124B. The crossmember 122 and the legs 124A, 124B form a trapezoidal shape with a first end 126A at the crossmember 122, a first lateral side formed by leg 124A, a second lateral side formed by leg 124B, and a second end 126B at the opening between the ends of the legs 124A, 124B opposite the crossmember 122. The crossmember 122 and the first end 126A of the trapezoidal shape are positioned proximal to the post 114, while the opening between opposite ends of the legs 124A, 124B are positioned distal to the post 114. Legs 124A and 124B both extend from the crossmember 122 at an angle, such that the legs 124A, 124B extend away from each other. Thus, the second end 126B of the trapezoidal shape of the shield 120 is wider than the first end 126A. In the illustrated implementation, the shield 120 generally forms an open structure formed by the crossmember 122 and the legs 124A, 124B at the edges.

As shown in FIG. 2, when the drape 130 is coupled to or overlaid on the shield 120, an interior space 132 is defined by the shield 120 and the drape 130. During use, the patient 200 (and the face 201 of the patient) is disposed within the interior space 132. The shield 120 extends upward and outward from the post 114 towards the patient 200 such that a central portion of the shield 120 between the first end 126A and the second end 126B is generally aligned with the face 201 of the patient 200.

The drape 130 generally includes at least one opening sized to allow a healthcare worker to reach their hands into the interior space 132 defined by the shield 120 and the drape 130, wherein the face 201 of the patient 200 is located. In the illustrated implementation, the drape 130 includes two openings 134A, 134B defined on either side of the drape 130. Opening 134A is thus spaced apart from the post 114 in a first direction (which is generally lateral and outward), while opening 134B is spaced apart from the post 114 in a second direction (which is also generally lateral and outward). During use, the healthcare worker (e.g., a doctor, a nurse, etc.) generally stands with their torso in line with the vertical post 114 and the patient 200, such that they are able to reach their left hand into the interior space 132 through opening 134A, and their right hand into the interior space 132 through opening 134B.

In additional or alternative implementations, the drape 130 includes flap portions that overlay the openings 134A, 134B when the drape 130 is not being used and the healthcare worker is not extending their arms through the openings 134A, 134B. When the healthcare worker extends their arms through the openings 134A, 134B, the flaps move out of the way to allow access to the patient 200. The flaps can thus aid in in sealing off the interior space 132 prior to the healthcare worker extending their arms through the openings 134A, 134B. In other implementations, the drape 130 can include gasket-type valves at the openings 134A, 134B (formed from rubber, latex, etc.) that maintain a seal when the healthcare worker does not have his or her arms extended through the openings 134A, 134B.

Because the shield 120 extends upward and outward from the post 114, and because the legs 124A, 124B of the shield 120 extend outward from the crossmember 122 away from each other, the width and height of the interior space 132 both increase from a first end proximal to the post 114, to a second end distal from the post 114. The interior space 132 defined by the shield 120 and the drape 130 thus has a large amount of volume. This large volume allows the healthcare worker a sufficient amount of room to maneuver their arms and hands, in order to intubate the patient 200 or perform other procedures. In particular, because the post 114 is generally positioned directly in front of the healthcare worker's torso, and because the legs 124A, 124B extend upward and outward from the healthcare worker, the amount of obstruction experienced by the healthcare worker due to rigid portions of the device 100 is minimized. Generally, the only portion of the device 100 that contacts the healthcare worker during use is the drape 130. However, because the drape 130 is formed from a flexible material, contact between the healthcare worker and the drape 130 minimally interferes with the intubation procedure.

Further, the drape 130 extends on all sides of the patient 200 (including between the patient 200 and the torso of the healthcare worker). Because the face 201 of the patient 200 is positioned in the interior space 132, at least a portion of the drape 130 is positioned between the healthcare worker and the face 201 of the patient 200. The healthcare worker is thus protected from any respiratory droplets or other bodily fluids that may be emitted from the patient 200 during the intubation procedure.

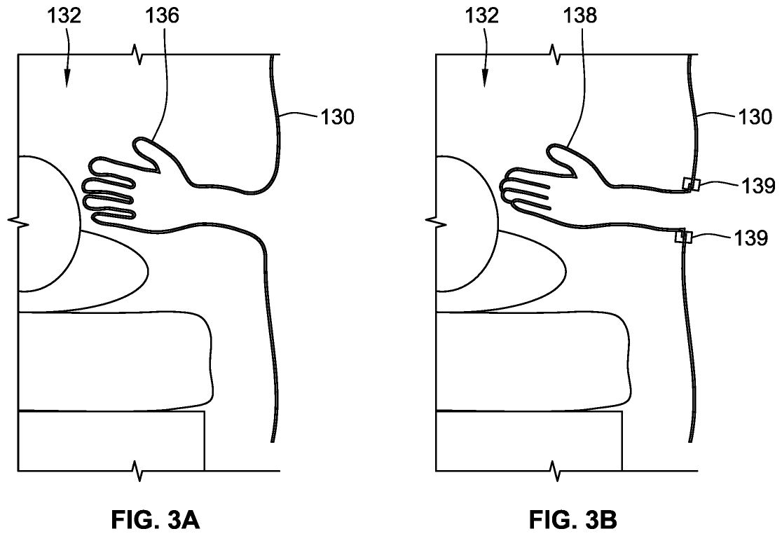

FIG. 3A and FIG. 3B show cross-sectional views of additional implementations that allow a healthcare worker to access the interior space 132 defined by the drape 130. FIG. 3A shows an implementation where the drape 130 forms an inwardly-facing glove 136. The glove 136 is made from the same material 130 as the drape, and is generally formed continuously with the drape 130. FIG. 3B shows an implementation where a separate inwardly-facing glove 138 is sealed to the inside of the drape 130. In FIG. 3B, the glove 138 is sealed to the drape 130 via a seal 139. Because FIG. 3B is a cross-sectional view, seal 139 appears in FIG. 3B with an upper portion and a separate lower portion. However, seal 139 will generally have a circular shape that matches the opening of the glove 138. The glove 138 could be made from a suitable material, such as rubber, latex, etc., and can be sealed to the drape 130 after the drape 130 and the glove 138 are formed. While FIGS. 3A and 3B show a single glove 136 and a single glove 138, it is understood that these implementations will generally include one glove 136/138 on both sides of the drape 130, so that the healthcare worker can insert both of their hands into a glove.

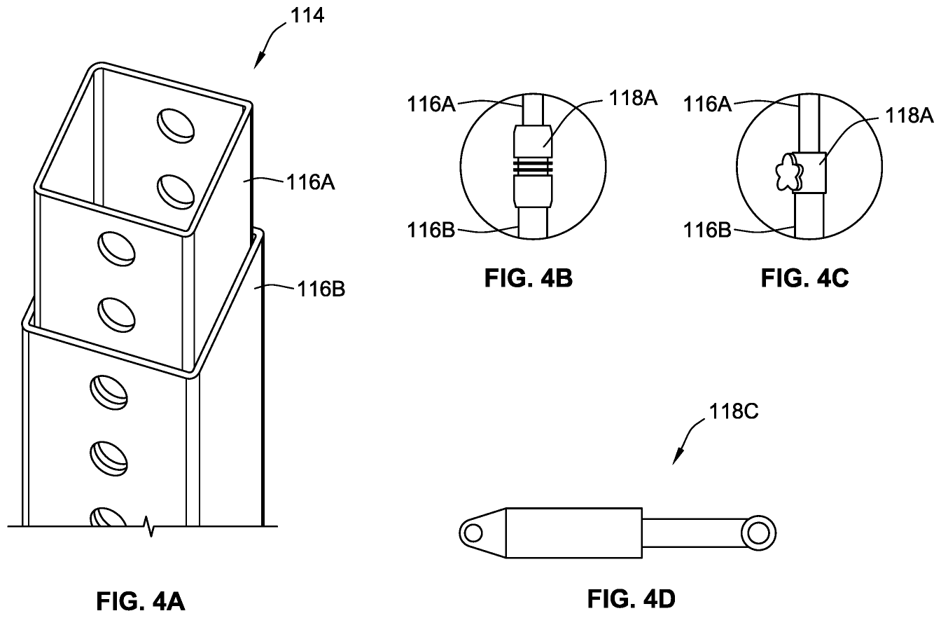

Referring now to FIG. 4A, the post 114 of the base 110 can be height-adjustable, to provide more flexibility for different setups and different patients. In some implementations, the post 114 includes an inner post 116A that is coaxially mounted inside an outer post 116B. In other implementations, the two posts 116A, 116B could be mounted next to each other. Generally, either the inner post 116A or the outer post 116B are coupled to the baseplate 112, and the other of is coupled to the shield 120. The inner post 116A and the outer post 116B can move relative to each other, such that the height of the shield 120 relative to the baseplate 112 can be adjusted. This allows the device 100 to be used with larger and smaller patients, and also effectively work with different setups that may affect the height of the shield 120. For example, if the baseplate 112 is coupled to some structure closer to the ground (such as the bottom of a bed frame), the shield 120 will be in a lower position as compared to if the baseplate 112 is anchored by the mattress of the bed or the patient. The interior space 132 thus might be too small to effectively allow the healthcare worker to intubate the patient. By using a height-adjustable post 114, the height of the shield 120 relative to the baseplate 112 can be modified to work in both situations. In some implementations, the shield 120 is raised to such a height during use that the drape 130 is approximately 18 inches above the face 201 of the patient 200, and such that the top of the post 114 is approximately at the mid-chest level of the healthcare worker performing the procedure.

In some implementations, the two posts 116A, 116B are manually movable by a user. In additional or alternative implementations, the posts 116A, 116B can be moved via a hydraulic mechanism, an electronic mechanism, or other mechanisms.

FIGS. 4B-4D illustrates a variety of different example mechanisms that can be used to lock the inner post 116A and the outer post 116B in place. When engaged, movement of the inner post 116A and the outer post 116B relative to each other is prevented. In FIG. 4B, a mechanism 118A is coaxially mounted to the outer post 116B, and can be rotated along an axis parallel to the inner posts 116A and 116B to lock or unlock the inner post 116A and the outer post 116B. In FIG. 4C, a mechanism 118B is mounted to the outer post 116B, and can be rotated along an axis perpendicular to the inner posts 116A and 116B to lock or unlock the inner post 116A and the outer post 116B. In FIG. 4D, a hydraulic mechanism 118C can be coupled to the posts 116A and 116B, and can be operated to cause relative movement between the two posts 116A, 116B.

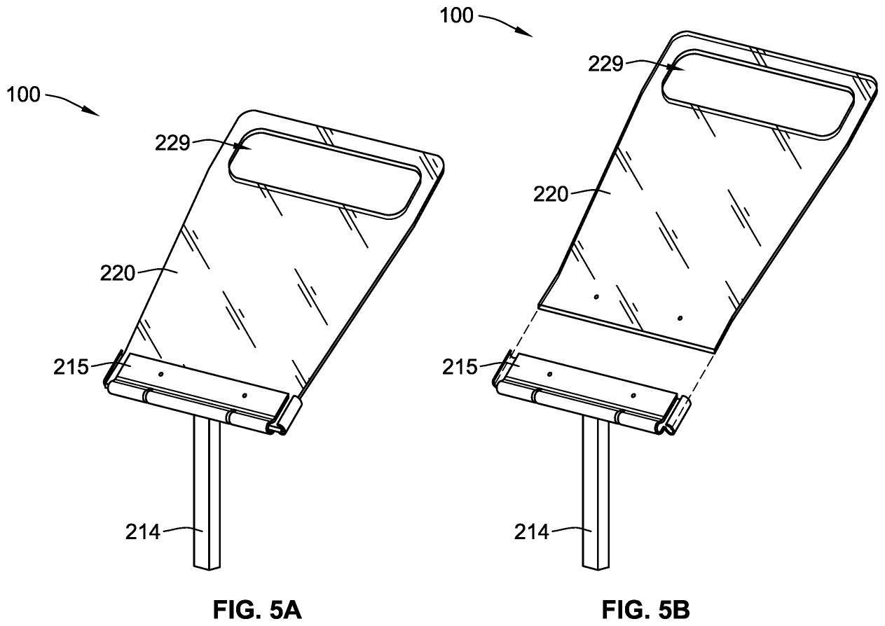

FIGS. 5A and 5B illustrate an alternative implementation of the device 100 with a modified shield. FIG. 5A shows a shield 220 coupled to an attachment point 215 of a post 214. FIG. 5B shows the shield 120 decoupled from the attachment point 215. As can be seen, the shield 220 can easily be detached from the post 214, for example if the shield 220 needs to be repaired or discarded. A drape (such as drape 130) can be placed over the shield 220, similar to how the drape 130 is placed over the shield 120.

The shield 220 forms a closed shape, in contrast to the open shape of the shield 120. In FIGS. 5A and 5B, the shield 220 has a generally rectangular shape. However, the shield 220 could have any suitable shape in these implementations, so long as the shape is closed. In these implementations, the shield 220 can define a cut-out portion 229 that forms a handle. This handle allows a user to easily grasp the shield 220, in order to attach the shield 220 to the attachment point 215 of the post 214, and detach the shield 220 from the attachment point 214 of the post 214. The cut-out portion 229 also reduces the weight of the shield 220, which can reduce any wobbling of the shield 220 that may be present due to the cantilevered nature of the post 214 and the shield 220.

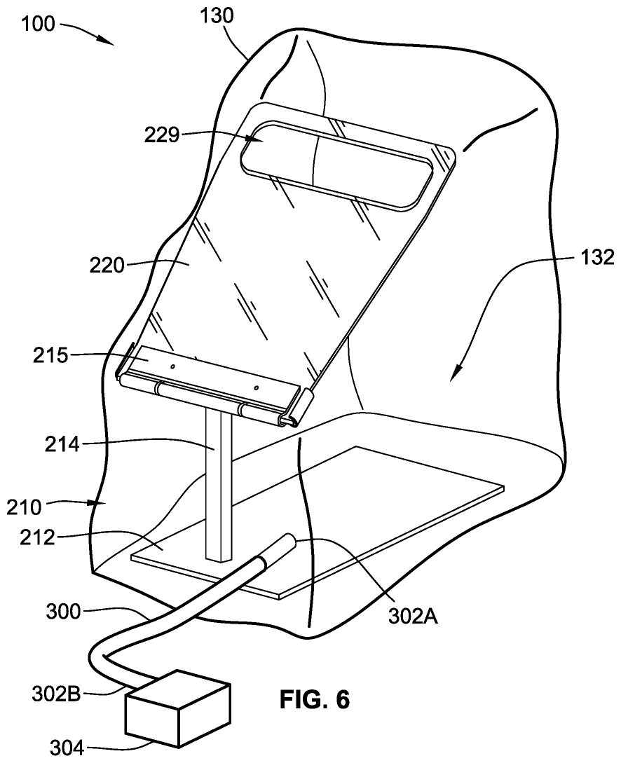

FIG. 6 illustrates the device 100 and the shield 220 with the drape 130 placed over top. As shown, the shield 220 (which includes the cut-out portion 229) is coupled to a base 210, which includes a baseplate 212, the post 214 and the attachment point 215. FIG. 6 also shows a tube 300 that is extends through the drape 130. A first end 302A of the tube 300 is disposed within the interior space 132 defined by the drape 130. A second end 302B extends outside of the drape 130, and is coupled to an air pump 304. The tube 300 thus fluidly couples the interior space 132 to the pump 304. The tube 300 and the pump 304 can be used to remove unwanted particles from the interior space 132 during use. For example, while a healthcare worker is using the device 100 to intubate a patient, the interior space 132 can begin to fill up with respiratory droplets, bacteria particles, viral particles, or generally any other unwanted substance or material. If these particles build up within the interior space 132, the healthcare worker can be exposed to a potentially dangerous concentration of these particles when the device 100 is removed after the patient has been intubated. The pump 304 can be operated while the device 100 is placed over the patient, to gradually remove any unwanted particles from the interior space 132 through the tube 300. The tube 300 is generally sealed to the drape 130, so that there are no leaks where the tube 300 passes through the drape 130. One or more filters may also be located within the tube 300, within the pump 304, and/or adjacent to the pump 304, that are configured to trap the unwanted particles.

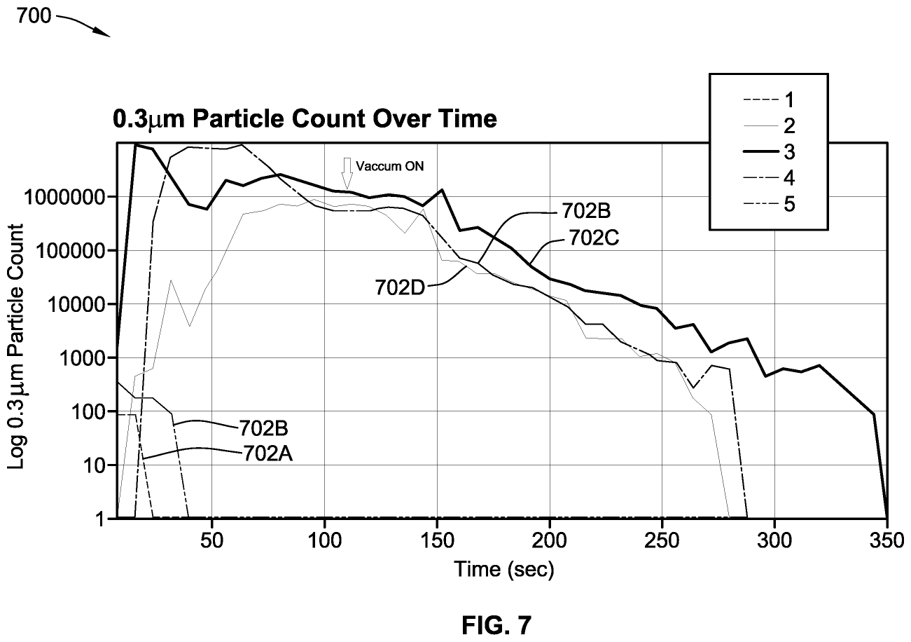

FIG. 7 shows a plot 700 demonstrating the efficacy of the removal of particles from within the interior space 132 of device 100, which could be used with shield 120 (FIGS. 1A-2) or shield 220 (FIGS. 5A-6). The horizontal axis shows the time measured in seconds, and the vertical axis shows the logarithm of the count of 0.3 μm particles within the interior space 132 defined by the flexible drape 130. To obtain the plot 700, a smoke generator was placed within the interior space 132 formed by the drape 130, and a particle counter was used to measure the amount of 0.3 μm smoke particles at various different locations of the device 100 over time. The measurements were made in a positive pressure environment with airflow outside of the device 100.

Data series 702A corresponds to measurement location 1, which is located outside of the drape 130, near where the healthcare worker's head would be during use. Data series 702E corresponds to measurement location 5, which is located outside of the drape 130, near the distal end of the shield 120, spaced apart from where the healthcare worker's head would be during use. Data series 702B, 702C, and 702D correspond to measurement locations 2, 3, and 4, respectively. Each of these measurement locations are located inside of the drape 130. Thus, data series 702A-702E show the particle counts at various locations inside and outside of the drape 130.

The smoke generator was activated at a time of 0 seconds. As can be seen, data series 702A and 702E quickly drop to near zero, indicating that the particle count at the two locations outside of the drape 130 were almost immediately zero. In contrast, the particle counts shown by data series 702B, 702C, and 702D increased and remained high once the smoke generator was activated. Then, once the pump was activated (at a time between 100 seconds and 150 seconds) and substances from within the drape 130 were extracted via the tube, the particle counts at the three measured locations inside of the drape 130 began to decrease. The particle counts at locations 2 and 4, illustrated by data series 702B and 702D, fell to zero between 250 seconds and 300 seconds. The particle count at location 3, illustrated by data series 702C, fell to zero around 350 seconds. Thus, plot 700 illustrates that the device 100 is effective at preventing unwanted substances emitted by the patient (e.g., respiratory droplets, viral particles) from reaching healthcare workers positioned outside of the drape 130, and allows for the unwanted substances to be removed from the inside of the drape 130 using the tube and the air pump, prior to the device 100 being completely removed from the patient once the intubation procedure is completed.

In some implementations, one or both of the shield (include shield 120 or shield 220) and the flexible drape 130 are disposable, such that the shield 120/220 and the drape 130 can be discarded after use. In other implementations, the shield 120/220 and the drape 130 can be reused after being thoroughly cleaned and disinfected. In some implementations, the shield 120/220 and/or the flexible drape 130 can be made from any combination of polycarbonate, acrylic, polyethylene, terephthalate glycol, clear vinyl, or other clear plastic sheeting.

While FIG. 2 shows the device 100 being used in a hospital setting, the device 100 can generally be used in any suitable setting. For example, a first responder may use the device 100 in the field to intubate a patient (or perform other procedures on the patient). This can be helpful in situations where it is unknown if the patient has any communicable diseases or conditions. The device 100 can also be used in an emergency room setting, where it is also common that it is unknown if the patient has any communicable diseases.

Because of the flexibility of usable settings, the device 100—specifically the baseplate 112 and the post 114/214—is readily adaptable to a variety of scenarios. The baseplate 112/212 and/or the post 114 are generally configured to be attached to any suitable mounting point, which can include underneath a mattress, underneath a patient's head, a portion of a bed frame of the patient's bed, a gurney, a backboard, etc. In some implementations, the baseplate 112/212 is weighted, such that the device 100 can be used without attaching the baseplate 112/212 to a separate structure.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Numerous changes to the disclosed embodiments can be made in accordance with the disclosure herein, without departing from the spirit or scope of the invention. Thus, the breadth and scope of the present invention should not be limited by any of the above described embodiments. Rather, the scope of the invention should be defined in accordance with the following claims and their equivalents.

Although the invention has been illustrated and described with respect to one or more implementations, equivalent alterations, and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

1. A device for shielding a user from a patient during an intubation procedure or other procedure, the device comprising:
a base;
a shield coupled to the base, the shield extending vertically and horizontally relative to the base; and
a flexible drape coupled to or overlaid on the shield such that an interior space is defined by the shield and the flexible drape, the flexible drape including at least one opening to allow access into the interior space.

2. The device of claim 1, wherein the shield is transparent.

3. The device of claim 1, wherein the shield has a trapezoid shape with a first end, a second end, a first lateral side, and a second lateral side, the first end being positioned proximal to the base, the second end being positioned distal to the base.

4. The device of claim 3, wherein the device is configured to be positioned relative to the patient such that a central portion of the shield between the first end and the second end is aligned with the face of the patient.

5. The device of claim 3, wherein the second end of the shield is longer than the first end of the shield, such that a width of the shield increases between the first end and the second end.

6. The device of claim 1, wherein the at least one opening of the flexible drape includes a first opening on a first side of the drape and a second opening on a second side of the drape opposite the first side of the drape, the first opening, the second opening, or both being sized to allow an arm of the user to access the interior space.

7. (canceled)

8. The device of claim 1, wherein the base includes a generally horizontal baseplate and a generally vertical post coupled to the baseplate the shield being coupled to the generally vertical post.

9. (canceled)

10. The device of claim 8, wherein a height of the post is adjustable, such that a vertical distance between the shield and the baseplate is adjustable.

11. The device of claim 8, wherein the baseplate is configured to be positioned beneath a head of the patient, beneath a mattress the patient is lying on, or beneath a portion of a bed frame the patient is lying on.

12. The device of claim 1, wherein the base includes a slot configured to receive an end of the shield, and a shield-locking feature configured to lock the end of the shield in the slot.

13. (canceled)

14. The device claim 1, wherein the base includes a vertical post configured to be positioned between the user and a face of the patient, and wherein the at least one opening in the flexible drape includes a first opening spaced apart from the vertical post in a first direction, and a second opening spaced apart from the vertical post in a second direction, the first opening being sized to accommodate a first arm of the user, the second opening being sized to accommodate a second arm of the user.

15. The device of claim 1, wherein the shield is positioned at an angle of about 45° relative to horizontal, wherein the shield includes a cut-out portion forming a handle, or both.

16. (canceled)

17. The device of claim 1, wherein at least a portion of the shield is configured to be positioned between the user and a face of the patient, at least a portion of the flexible drape is configured to be positioned between the user and a face of the patient, or both.

18. (canceled)

19. The device of claim 1, wherein a face of the user is configured to be positioned in the interior space.

20. The device of claim 1, wherein a width of the interior space increases from a first end of the interior space proximal to the base to a second end of the interior space distal to the base.

21. The device of claim 1, wherein a height of the interior space increases from a first end of the interior space proximal to the base to a second end of the interior space distal to the base.

22. The device of claim 1, wherein the base includes a generally horizontal baseplate, a first generally vertical post, and a second generally vertical post, the first post coupled to the baseplate and the second post coupled to the shield, the first post and the second post being movable relative to each other such that a height of the shield is adjustable relative to the baseplate.

23. The device of claim 22, wherein the first post is coaxially mounted within the second post, or wherein the second post is coaxially mounted within the first post, and wherein the device further comprises a locking mechanism configured to lock relative movement between the first post and the second post.

24. (canceled)

25. The device of claim 1, wherein the base includes a generally horizontal baseplate and a generally vertical post, the shield being coupled to the vertical post such that an angle of the shield relative to the vertical post is adjustable.

26. The device of claim 1, further comprising:
a tube coupled to the flexible drape, the tube having a first end positioned within the interior space, and a second end positioned outside of the interior space; and
a pump positioned outside of the interior space and coupled to the tube such that the tube fluidly couples the pump to the interior space, the pump being configured to be operated to remove substances from within the interior space defined by the shield and the flexible drape via the tube.

27-28. (canceled)
